(19) United States
(12) Patent Application Publication
(10) Pub. No.: US 2012/0214043 A1
(43) Pub. Date: Aug. 23, 2012

(54) Lithium sulfur battery

(75) Inventors: Jens Olschimke, Hannover (DE); Martin Bomkamp, Hannover (DE); Johannes Eicher, Sehnde (DE)

(73) Assignee: SOLVAY FLUOR GMBH, Hannover (DE)

(21) Appl. No.: 13/504,094

(22) PCT Filed: Oct. 26, 2010
(86) PCT No.: PCT/EP10/66143

§ 371 Date: Apr. 25, 2012

(30) Foreign Application Priority Data
Oct. 27, 2009 (EP) 09174210.6

(51) Int. Cl. H01M 2/16; H01M 10/056; C07C 69/96

(52) U.S. Cl. 429/144; 558/260; 429/338; 429/342; 429/343

(57) ABSTRACT

A lithium sulfur battery comprising an electrolyte solvent which comprises at least one fluorosubstituted compound is described. Preferred fluorosubstituted compounds which are predominantly solvents are notably selected from the group consisting of fluorosubstituted carboxylic acid esters, fluorosubstituted carboxylic acid amides, fluorosubstituted fluorinated ethers, fluorosubstituted carbamates, fluorosubstituted cyclic carbonates, fluorosubstituted acyclic carbonates, fluorosubstituted ethers, perfluoroalkyl phosphoranes, fluorosubstituted phosphites, fluorosubstituted phosphates, fluorosubstituted phosphonates, and fluorosubstituted heterocycles. Monofluoroethylene carbonate, cis-difluoroethylene carbonate, trans-difluoroethylene carbonate, 4,4-difluoroethylene carbonate, trifluoroethylene carbonate, tetrafluoroethylene carbonate, 4-fluoro-4-methyl-1,3-dioxolane-2-one, 4-fluoro-4-ethyl-1,3-dioxolane-2-one, 2,2,2-trifluoroethyl-methyl carbonate, 2,2,2-trifluoroethyl-fluoromethyl carbonate are preferred. The solvent may further comprise a non-fluorinated solvent, e.g., ethylene carbonate, a dialkyl carbonate, or propylene carbonate. Use of such fluorinated compound as additive for such batteries and specific electrolyte solutions.

### CROSS-REFERENCE TO RELATED APPLICATIONS

This application is a U.S. national stage entry under 35 U.S.C. §371 of International Application No. PCT/EP2010/066143 filed Oct. 26, 2010, which claims priority benefit to European Patent Application No. 09174210.6 filed on Oct. 27, 2009, the whole content of this application being incorporated herein by reference for all purposes.

### TECHNICAL FIELD OF THE INVENTION

The present invention concerns a Li—S battery, the use of fluorinated additives for LiS batteries, and novel electrolyte solutions.

### BACKGROUND OF THE INVENTION

Lithium sulfur batteries, or shortly in the context of the present invention, “Li—S” batteries, are applicable as rechargeable power sources for a lot of devices, for example, for computers, cell phones, and many other electric components. They have a high theoretical specific energy of 2600 Wh/kg, and sulfur is relatively non-toxic. The battery is also a suitable energy source for electrically driven vehicles.

The underlying principle is that they comprise a metal anode in an anode compartment and a cathode (for example, porous carbon), with a gel-polymer electrolyte membrane or a non-aqueous solvent which serve as ion-transporting medium. Often, a membrane—for example, a LISICON membrane or a NASICON membrane—which is permeable for the metal cation, but impermeable for other compounds, serves to effectively separate anode and cathode compartments. The problem of the present invention is to provide a Li—S battery. Another problem is to provide electrolyte solutions suitable for Li—S batteries. These and other problems are solved by the battery, the electrolyte solutions and the use of fluorosubstituted organic compounds as stated in the claims.

### SUMMARY OF THE INVENTION

According to the present invention, a Li—S battery is provided comprising an electrolyte solvent which comprises or consists of at least one fluorosubstituted organic compound which contains at least one heteroatom selected from the group consisting of oxygen, nitrogen, phosphorous, sulfur and silicon, and wherein the fluorosubstituted organic compound is selected from the group consisting of fluorosubstituted carboxylic acid esters, fluorosubstituted carboxylic acid amides, fluorosubstituted fluorinated ethers, fluorosubstituted carbamates, fluorosubstituted cyclic carbonates, fluorosubstituted acyclic carbonates, fluorosubstituted ethers, perfluoroalkyl phosphoranes, fluorosubstituted phosphites, fluorosubstituted phosphates, fluorosubstituted phosphonates and fluorosubstituted heterocycles. The term “Li—S battery” has the same meaning as “lithium sulfur battery”.

### BRIEF DESCRIPTION OF THE DRAWING

For a detailed description, reference will now be made to the accompanying drawing, in which:

FIG. 1 shows a lithium-sulfur battery and an apparatus which is powered by the electric current provided by the battery.

### DETAILED DESCRIPTION

The electrolyte solvent is selected such that it is liquid at the temperature at which the battery is intended to be used. If the melting point of the respective fluorosubstituted organic compound is low enough it can be used neat. The melting point of monofluoroethylene carbonate (“FlEC”) is about 22° C. Thus, it is preferred to apply this compound with a co-solvent with a lower melting point, e.g., with dimethyl carbonate or diethyl carbonate which have a melting point of about 2 to 4° C., with ethyl methyl carbonate having a melting point of −14.5° C., or with propylene carbonate with a melting point in the range of −50° C. Thus, in this case, the solvent is a solvent mixture.

The Li—S battery of the present invention preferably comprises a membrane permeable for the metal cations and comprises a thin, dense, substantially non-porous layer which is sandwiched by porous layers.

The term “metal” denotes lithium.

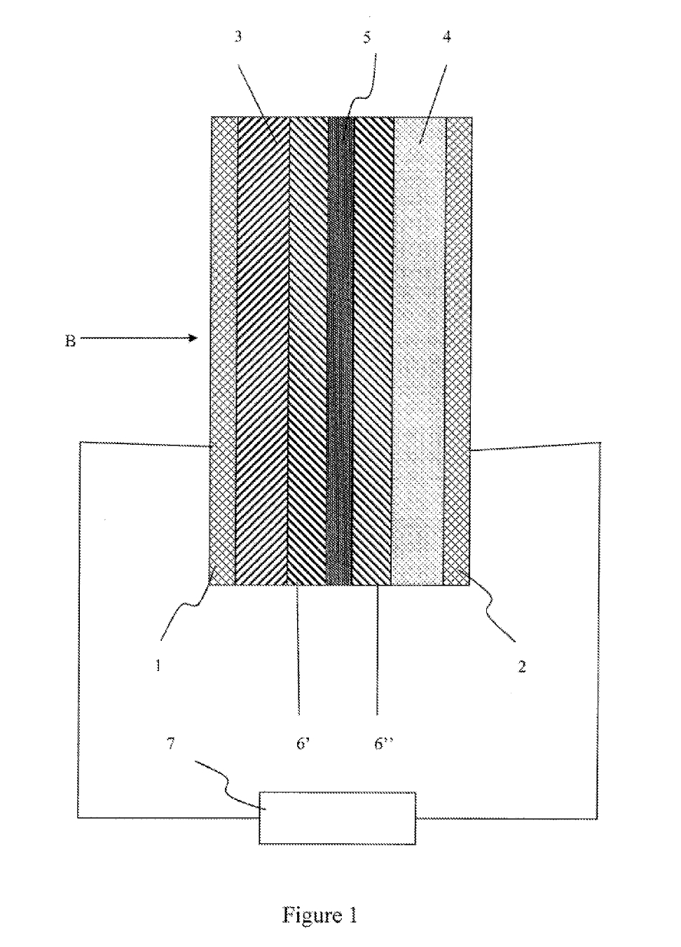

A metal-air battery is disclosed in U.S. Pat. No. 5,510,209. The metal is lithium, magnesium, sodium, calcium, aluminium or zinc. The battery described therein, for example, in FIG. 1 of U.S. Pat. No. 5,510,209, comprises a lithium foil anode, a polymer electrolyte (comprising polyacrylnitrile, a solvent, e.g., propylene carbonate or ethylene carbonate, and an electrolyte salt, e.g., LiPF₆), a composite positive electrode current collector and an oxygen permeable membrane through which oxygen (from the surrounding air) is transported to the porous carbon electrode.

The battery according to the present invention provides a lithium sulfur battery the solvent of which consists or comprises a fluorosubstituted organic compound as defined above. A polymer can be present in the electrolyte, but it is not necessarily present in the battery of the current invention; it is sufficient to provide the lithium ion transporting solvent or solvent mixture which contains or consist of a fluorosubstituted organic compound.

The invention will now be described in further detail in view of the Li—S battery.

Preferably, the electrolyte solvent is liquid at a temperature equal to or above −20° C.

FIG. 1 describes a very suitable lithium battery B. The battery B comprises current collectors 1 and 2. The anode 3 comprises lithium metal. The cathode 4 comprises elemental sulfur, Li₂Sₓ and a fluorosubstituted solvent. A thin, dense, substantially non-porous layer 5 is sandwiched by porous layers 6′ and 6″. Non-porous layer 5 and porous layers 6′, 6″ may be a composite LISICON membrane. The battery is connected to an apparatus 7 (could be for example a cell phone) which is powered by the electric current provided by the battery B.

The chemical processes in the Li—S cell include lithium dissolution from the anode surface during discharge, and lithium plating back on to the nominal anode while charging.

On the anode, Li is oxidized forming Li⁺. On the cathode, sulfur is reduced to polysulfides and finally to Li₂S:

S₈→Li₂S₈→Li₂S₆→Li₂S₄→Li₂S

Upon charging the lithium sulfur battery, the reverse reactions happen. The Li₂S is broken at the cathode to produce finally elemental sulfur:

Li₂S→Li₂S₂→Li₂S₃→Li₂S₄→Li₂S₆→Li₂S₈→S₈.

The Li⁺ ions pass to the anode to be reduced to Li metal.

It is clear for the expert that a single fluorosubstituted organic compound can be applied or a mixture of two or more fluorosubstituted organic compounds. In the context of the present invention, the singular form “fluorosubstituted organic compound” is intended to include the plural, i.e. a mixture of two or more fluorosubstituted organic compounds.

According to an embodiment of the present invention, the fluorosubstituted organic compound can be applied as electrolyte solvent or as component of the electrolyte solvent of Li—S batteries in which the anode is in contact with the electrolyte solvent. In such a type of battery, often reactions of the anode with the solvent are observed, or Li dendrites grow on the anode and sooner or later give rise to short cuts.

According to a preferred embodiment, the Li—S battery is of the type which contains a membrane between the anode and the cathode compartment. The invention will now be explained in further detail in view of this preferred embodiment. The anode contains lithium and the cathode comprises elemental sulfur and at least one solvent selected to at least partially dissolve the elemental sulfur and Li₂Sₓ. A substantially non-porous lithium-ion conductive membrane is provided between the anode and the cathode to keep sulfur and other reactive species from migrating between the anode and cathode. The non-porous membrane is for example a thin ceramic membrane. Li—S batteries in which solvents are used to dissolve sulfur and lithium sulfide and lithium polysulfides and which comprise a membrane between the anode and cathode compartments are described in US patent application publication 2009/0061288 which is incorporated herein by reference in its entirety for all purposes. Sulfur which is apolar dissolves in an apolar solvent such as benzene, fluorobenzene, toluene, trifluorotoluene, xylene, cyclohexane, tetrahydrofurane or 2-methyl tetrahydrofurane. Lithium sulfide and lithium polysulfides are polar compounds and thus dissolve in polar solvents such as a carbonate organic solvent or tetraglyme.

The fluorosubstituted organic compound is selected such that it does not react in undesired manner with Li⁺ ions, with sulfur and with any of the lithium sulfides and lithium polysulfides formed. The compatibility of a suitable fluorinated organic compound can be identified by a test, e.g., by testing a respective battery in a certain number of charge-discharge cycles, controlling voltage and capacity.

In the following, preferred fluorinated organic solvents are presented. As mentioned above, these compounds can be applied in admixture with other solvents, for example, non-halogenated solvents, or solvents which are chlorinated or they can constitute the sole solvent or solvents of the cathode compartment. Preferably, solvents are applied which are not substituted by chlorine atoms.

The solvents can be applied in batteries with liquid electrolytes and in batteries with gel state electrolytes. In gel state electrolytes, the non-aqueous solvents are gelled through the use of a gelling agent such as polyacrylonitrile, polyethylene oxide, polyvinylidene fluoride. Polymerizable monomers that are added to the non-aqueous solvent system and polymerized in situ by the use of heat or radiation may also be used.

Preferred fluorinated organic compounds are selected from the group consisting of monofluorinated, difluorinated, trifluorinated, polyfluorinated and perfluorinated organic compounds. Here, the term “polyfluorinated” denotes compounds which are substituted by four or more fluorine atoms, but contain at least one hydrogen atom, or at least one chlorine atom, or at least one hydrogen atom and at least one chlorine atom. Preferably, the monofluorinated, difluorinated, trifluorinated, polyfluorinated and perfluorinated organic compounds are not substituted by chlorine atoms. Perfluorinated are those compounds in which all hydrogen atoms are substituted by fluorine atoms.

Preferred fluorinated organic compounds are selected from the group of fluorosubstituted carboxylic acid esters, fluorosubstituted carboxylic acid amides, fluorosubstituted fluorinated ethers, fluorosubstituted carbamates, fluorosubstituted cyclic carbonates, fluorosubstituted acyclic carbonates, fluorosubstituted phosphites, fluorosubstituted phosphoranes, fluorosubstituted phosphoric acid esters, fluorosubstituted phosphonic acid esters and saturated or unsaturated fluorosubstituted heterocycles.

Suitable fluorinated ethers are for example those as described in U.S. Pat. No. 5,916,708, namely partially fluorinated ethers of formula (I)

RO—[(CH₂)ₘO]ₙ—CF₂—CFH—X (I)

wherein
R is a linear alkyl group with 1 to 10 C atoms or a branched alkyl group with 3 to 10 C atoms,
X is fluorine, chlorine or a perfluoroalkyl group with 1 to 6 C atoms which groups may include ether oxygen,
m is an integer of from 2 to 6 and
n is an integer of from 1 to 8,
and/or of formula (II)

X—CFH—CF₂O—[(CH₂)ₘO]ₙ—CF₂—CFH—X (II)

wherein
X, m and n have the meaning given above.

Suitable partially fluorinated carbamates are for example those described in U.S. Pat. No. 6,159,640, namely compounds of the formula R¹R²N—C(O)OR³ wherein R¹ and R² independently are the same or different, and are linear C1-C6-alkyl, branched C3-C6-alkyl, C3-C7-cycloalkyl, or R¹ and R² are connected directly or via one or more additional N and/or O atoms forming a ring with 3 to 7 members. Optionally, additional N atoms in the ring are saturated with C1 to C3 alkyl groups, and additionally, the carbon atoms of the ring may be substituted by C1 to C3 alkyl groups. In the groups R¹ and R², one or more hydrogen atoms may be substituted by fluorine atoms. R³ is a partially fluorinated or perfluorinated linear or branched alkyl group with 1 to 6 or, respectively, 3 to 6 carbon atoms, or a partially or perfluorinated cycloalkyl group with 3 to 7 C atoms, which may be substituted by one or more C1 to C6 alkyl groups.

Suitable fluorinated acetamides are for example those described U.S. Pat. No. 6,489,064, namely partially fluorinated amide corresponding to formula (I)R¹CO—NR²R³ (III) wherein R¹ is a linear C1-C6 alkyl group in which at least one hydrogen atom is replaced by fluorine, or a branched C3-C6 alkyl group in which at least one hydrogen atom is replaced by fluorine, or a C3-C7 cycloalkyl group optionally substituted one or more times by a linear C1-C6 alkyl group or branched C3-C6 alkyl group or both in which at least one hydrogen atom of the cycloalkyl group or the optional linear or branched alkyl substituent or both is replaced by fluorine, and R² and R³ independently represent an identical or different linear C1-C6 alkyl group, a branched C3-C6 alkyl group or a C3-C7 cycloalkyl group, or together with the amide nitrogen form a saturated five or six-membered nitrogen-containing ring, or are joined with one or more additional N and/or O atom(s) to form a 4 to 7-membered ring in which the additional N atoms present in the ring are optionally saturated with C1-C3 alkyl groups and the ring carbon atoms may also carry C1-C3 alkyl groups.

Suitable partially fluorinated esters are for example those described in U.S. Pat. No. 6,677,085 partially fluorinated compound derived from a diol corresponding to formula (IV): R¹CO—O—[CHR³(CH₂)ₘ—O_{]n}—R² (IV) wherein R¹ is a (C1-C8) alkyl group or a (C3-C8) cycloalkyl group, wherein each of said groups is partially fluorinated or perfluorinated so that at least one hydrogen atom of the group is replaced by fluorine; R² is a (C1-C8) alkyl carbonyl or (C3-C8) cycloalkyl carbonyl group, wherein said alkylcarbonyl or cycloalkylcarbonyl group may optionally be partially fluorinated or perfluorinated; R³ is a hydrogen atom or a (C1-C8) alkyl or (C3-C8) cycloalkyl group; m is 0, 1, 2 or 3, and n is 1, 2 or 3.

Especially preferred are linear or branched fluorosubstituted dialkyl carbonates and fluorosubstituted alkylene carbonates.

Suitable fluorinated dialkyl carbonates are those of formula (V)

R¹O—C(O)—O—R² (V)

In the compounds of formula (V), R¹ and R² can be the same or different with the proviso that at least one of R¹ and R² are substituted by at least one fluorine atom. R¹ and R² are preferably linear alkyl groups with 1 to 8 carbon atoms, preferably, 1 to 4 carbons, more preferably, with 1 to 3 carbon atoms; branched alkyl groups with 3 to 8 carbon atoms, preferably with 3 carbon atoms; or cyclic alkyl groups with 5 to 7 carbon atoms, preferably, 5 or 6 carbon atoms; with the proviso that at least one of R¹ and R² is substituted by at least one fluorine atom.

Highly preferably, R¹ and R² denote linear alkyl groups with 1 to 3 carbon atoms, with the proviso that at least one of R¹ and R² is substituted by at least one fluorine atom. Most preferably, R¹ and R² are selected from the group consisting of methyl, fluoromethyl, difluoromethyl, trifluoromethyl, ethyl, 1-fluoroethyl, 2-fluoroethyl, 1,1-difluoroethyl, 1,2-difluoroethyl, 2,2,2-trifluoroethyl and 1-fluoro-l-methylethyl. Most preferred compounds of formula (V) are methyl fluoromethyl carbonate, fluoromethyl ethyl carbonate, methyl 2,2,2-trifluoroethyl carbonate, fluoromethyl 2,2,2-trifluoroethyl carbonate and bis-2,2,2-trifluoroethyl carbonate. Such compounds can be manufactured from phosgene, COFCl or COF₂, and the respective alcohols, or as described in unpublished EP patent application No. 09155665.2. According to that process, the manufacture of fluoroalkyl (fluoro)alkyl carbonates of the general formula (Vi), FCHR—OC(O)—OR′ wherein R denotes linear or branched alkyl with 1 to 5 C atoms or H and R′ denotes linear or branched alkyl with 1 to 7 carbon atoms; linear or branched alkyl with 2 to 7 carbon atoms, substituted by at least one fluorine atom; phenyl; phenyl, substituted by 1 or more C1 to C3 alkyl groups atoms or phenyl substituted by 1 or more chlorine or fluorine atoms; or benzyl includes a step of reacting a fluoroalkyl fluoroformate of formula (VII), FCHROC(O)F, or a fluoroalkyl chloroformate of formula (VII′), FCHROC(O)Cl, with an alcohol of formula (VIII), R′OH, wherein R and R′ have the meanings given above, or includes a step of reacting a chloroalkyl fluoroformate of formula (IX), ClCHROC(O)F, or a chloroalkyl chloroformate of formula (IX′), ClCHROC(O)Cl, wherein R has the meaning given above, with an alcohol of formula (VIII), R′OH wherein R′ has the meaning given above, and a subsequent chlorine-fluorine exchange. The term “fluoro)alkyl” denotes alkyl and fluorosubstituted alkyl.

According to another embodiment, fluorosubstituted alkylene carbonates of formula (X) are applied

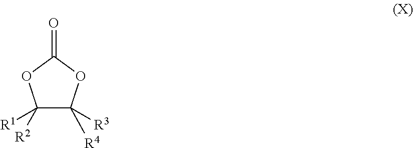

Here, R¹, R², R³ and R⁴ are independently selected from H, linear alkyl groups with 1 to 3 carbon atoms and alkenyl groups with 2 or 3 carbon atom; linear alkyl groups with 1 to 3 carbon atoms or an alkenyl group with 2 or 3 carbon atoms, substituted by at least one fluorine atom; and fluorine, with the proviso that at least one of R¹, R², R³ and R⁴ is fluorine or an alkyl group substituted by at least one fluorine atom.

According to one embodiment, in compounds of formula (X), R¹, R², R³ and R⁴ are selected from H and F, with the proviso that at least one of R¹, R², R³ and R⁴ is fluorine. Especially fluoroethylene carbonate, but also cis- and trans-4,5-difluoroethylene carbonate, 4,4-difluoroethylene carbonate, trifluoroethylene carbonate and tetrafluoroethylene carbonate are very suitable. These compounds can be manufactured by direct fluorination of ethylene carbonate. In the case of difluorosubstituted ethylene carbonate, cis and trans-4,5-difluoroethylene carbonate and 4,4-difluoroethylene carbonate are obtained. These isomers can be separated by fractionated distillation.

According to another preferred embodiment, in compounds of formula (X), R¹is a C1to C3 alkyl group or a C1 to C3 alkyl group, substituted by at least one fluorine atom; and R², R³ and R⁴ are H or F, with the proviso that at least one of R², R³ and R⁴ are F, or R¹is a C1 to C3 alkyl group, substituted by at least one fluorine atom. Preferably, R¹ is methyl, ethyl or vinyl.

Especially preferred compounds of this type are 4-fluoro-4-methyl-1,3-dioxolane-2-one, 4-fluoro-5-methyl-1,3-dioxolane-2-one, 4-ethyl-4-fluoro-1,3-dioxolane-2one, 5-ethyl-4-fluor-4-ethyl-1,3-dioxolan-2-one and 4,5-dimethyl-4-fluoro-1,3-dioxolane-2-one.

The compounds are known and can be manufactured by fluorination of the respective non-fluorinated compounds or by chlorine-fluorine exchange of the respective chlorosubstituted compounds. 4-Alkyl-4-fluorosubstituted compounds can be manufactured as described in 09161429.7: 4-fluoro-4-R-5-R′-1,3-dioxolane-2-ones are prepared by cyclization of compounds of formula (XI), FC(O)OCHR′C(O)R, wherein R is alkyl and R′ is H or C1 to C3 alkyl. R denotes preferably C1 to C5 alkyl, more preferably, C1 to C3 alkyl. Most preferably, R denotes methyl, ethyl, i-propyl and n-propyl. R′ preferably is H. Especially preferably, R is methyl and R′ is H.

The cyclization reaction is preferably catalyzed by a heterocyclic compound containing nitrogen, or by fluoride ions. In a preferred embodiment, the heterocyclic compound is an aromatic compound. For example, pyridine or 2-methylimidazole can be used as catalyst. Especially preferred are pyridines substituted by at least one dialkylamino group. 4-Dimethylaminopyridine is very suitable. Other 4-dialkylaminopyridines, for example, those wherein alkyl denotes a C1 to C3 alkyl group, are also considered to be suitable.

According to a further preferred embodiment, R¹ and R² are C1 to C3 alkyl groups or C1 to C3 alkyl groups, substituted by at least one fluorine atom; R³ and R⁴ are H or F, with the proviso that at least one of R³ and R⁴ are F, or at least one of R¹ and R² is a C1 to C3 alkyl group, substituted by at least one fluorine atom.

Especially preferred compounds of this type are 4-fluoro-5-(1-fluoroethyl)-1,3-dioxolan-2-one, 4-fluoro-5-(2-fluoroethyl)-1,3-dioxolan-2-one, 4-trifluoromethyl-4-methyl-1,3-dioxolan-2-one, 4-trifluoromethyl-4-methyl-5-fluoro-1,3-dioxolan-2-one and 4-(2,2,2-trifluoroethyl)-4-methyl-5-fluoro-1,3-dioxolan-2-one.

Another group of compounds are trialkyl phosphites wherein at least one alkyl group is substituted by at least one fluorine atom. Tris-(2,2,2-trifluoroethyl) phosphate is the preferred compound. It can be prepared from PCl₃ and trifluoroethanol, optionally in the presence of a base, e.g., an amine.

Still another group of compounds are perfluoroalkyl phosphoranes of formula (XII), (CnF₂n+m)₅P wherein n is 1, 2, 3, 4, 5, 6, 7 or 8, and m is +1 or −1. They can be prepared from pentaalkyl phosphanes via electrofluorination analogously to the process described in U.S. Pat. No. 6,264,818.

Fluorosubstituted phosphonate esters and phosphate esters of formula (XIII), R—P(O)R¹R², are also suitable. In formula (XIII), R is a C1 to C4 alkyl group; a C1 to C4 alkyl group, substituted by at least 1 fluorine atom; or a fluorosubstituted C2 to C4 alkoxy group; R¹ and R² are the same or different and represent C2 to C4 alkoxy groups, substituted by at least one fluorine atom. Preferred compounds of this type are methyl bis-(2,2,2-trifluoroethyl) phosphonate, ethyl bis-(2,2,2-trifluoroethyl) phosphonate, and tris-(2,2,2-trifluoroethyl) phosphate.

Fluorosubstituted carbonic acid esters of formula (XIV), R—C(O)OR¹ are also suitable. In the formula (XIV), R denotes preferably C1 to C3 and R¹ preferably denotes a C1 to C3 alkyl group with the proviso that at least one of R and R¹ are substituted by at least one fluorine atom. Preferred compounds are 2,2,2-trifluoroethyl butyrate (R=C₃H₇, R¹=C₂H₂F₃), ethyl trifluoroacetate (R=CF₃, R¹=C₂H₅), 2,2,2-trifluoroethyl acetate (R=CH₃, R¹=C₂H₂F₃) and methyl pentafluoropropionate (R=C₂F₅, R¹=CH₃). These compounds are suitable for batteries which are operated at low temperatures as described in US patent application publication 2008/0305401.

Another group of suitable compounds are those of formula (XV), R—C(O)—C(H)═C(H)—OR¹. In compounds of formula (XV), R is a polyfluorinated or perfluorinated alkyl group, and R¹ is C1 to C4 alkyl; C1 to C4 alkyl, substituted by one or more fluorine atoms; or phenyl. R is preferably CF₃, CHF₂, or C₂F₅; and R¹ is preferably methyl or ethyl. The most preferred compound is 4-Ethoxy-1,1,1-trifluoro-3-buten-2-one (ETFBO). These compounds can be prepared by the addition of the respective carboxylic acid chlorides to the respective vinyl ether and subsequent dehydrochlorination. ETFBO, for example, can be prepared from trifluoroacetyl chloride and ethyl vinyl ether. ETFBO is also available e.g., from Solvay Fluor GmbH, Hannover, Germany.

Another group of suitable compounds are polyfluorinated and perfluorinated ethers. Suitable perfluorinated polyethers are described, for example, in WO 02/38718. These perfluorinated polyethers consist essentially of carbon, fluorine and oxygen atoms and comprise at least two, preferably three, C—O—C ether linkages, or a mixture of several compounds satisfying that definition. Often, the oxygen atoms in the perfluoropolyether are exclusively present within the C—O—C ether linkages. The perfluoropolyethers generally have a molecular weight of about 200 or more. Generally they have a molecular weight of less than about 1500. If the polyether is a mixture of several substances, the molecular weight is the weight-average molecular weight. Generally, the perfluoropolyether has a boiling point greater than or equal to 40° C. at 101.3 kPa. The perfluoropolyether generally has a boiling point less or equal to about 200° C. at 101.3 kPa. As a result of the preparation, these perfluoropolyethers often are a mixture of individual substances. Generally, the kinematic viscosity of the perfluoropolyether is less than or equal to 1 cSt (Centistoke) at 25° C. Generally, the kinematic viscosity is at least 0.3 cSt at 25° C.

The preferred perfluoro polyethers are the products marketed by Solvay Solexis under the names GALDEN® and FOMBLIN®.

Examples include:

GALDEN HT 55: boiling point 57° C. at 101.3 kPa; average molecular weight 340
GALDEN HT 70: boiling point 66° C. at 101.3 kPa; average molecular weight 410
FOMBLIN PFS1: boiling point 90° C. at 101.3 kPa; average molecular weight 460

Partially fluorinated polyethers are the hydrofluoro ethers marketed by 3M under the name NOVEC®. The GALDEN® and FOMBLIN® systems are usually multicomponent systems having a boiling point in the range from 40 to 76° C.

Other fluorosubstituted compounds which are suitable as fluorosubstituted compound are lithium fluoro(oxalate)borate and lithium difluoro(oxalato)borate. They are no solvents but an electrolyte salt additive.

Also, fluorinated heterocycles are suitable, especially, fluorinated dioxolanes, fluorinated oxazolidines, fluorinated imidazolindines, fluorinated dihydroimidazoles, fluorinated 2,3-dihydroimidazoles, fluorinated pyrroles, fluorinated thiophenes, fluorinated thiazoles and fluorinated imidazoles.

Suitable fluorinated dioxolanes are for example 2,2-difluoro-1,3-dioxolane (U.S. Pat. No. 5,750,730) and 2-fluoro-4,4,5,5-tetramethyl-1,3-dioxolane, available from CHEMSTEP, France.

Suitable fluorinated oxazolidines are for example 2,2-difluoro-3-methyloxazolidine and 4,5-difluoro-3-methyloxazolidine-2-one, available from CHEMSTEP.

Suitable fluorinated imidazolidines are for example 2,2-difluoro-1,3-dimethylimidazolidine, available from ABCR GmbH & Co.KG, Karlsruhe, Germany, and 1,3-dibutyl-2,2-difluoroimidazolidineavailable from Apollo.

Suitable fluorinated 2,3-dihydroimidazoles are for example 2,2-difluoro-1,3-dimethyl-2,3-dihydro-1H-imidazole and 1-ethyl-2-fluoro-3-methyl-2,3-dihydro-1H-imidazole, available from CHEMSTEP.

Suitable fluorinated imidazoles are for example 1-(trifluoromethyl)-1H-imidazole, available from selectlab, and 2-fluoro-1-(methoxymethyl)-1H-imidazole, available from CHEMSTEP.

A suitable fluorinated pyrrole is for example 2-ethyl-5-fluoro-l-methyl-1H-pyrrole, available from CHEMSTEP.

A suitable fluorinated thiophene is for example 2-fluorothiophene, available from APAC Pharmaceutical.

A suitable fluorinated thiazole is for example 4-fluorothiazole, available from CHEMSTEP.

Also, fluorosubstituted organic liquids, e.g., 4,5-dimethyl-3-perfluorooctyl-1,2,4-triazolium tetrafluoroborate.

Other fluorosubstituted compounds which are members of the group consisting of fluorosubstituted carboxylic acid esters, fluorosubstituted carboxylic acid amides, fluorosubstituted fluorinated ethers, fluorosubstituted carbamates, fluorosubstituted cyclic carbonates, fluorosubstituted acyclic carbonates, fluorosubstituted ethers, perfluoroalkyl phosphoranes, fluorosubstituted phosphites, fluorosubstituted phosphates, fluorosubstituted phosphonates and fluorosubstituted heterocycles, or which preferably are present additionally to the F-substituted esters, amides, ethers, carbamates, cyclic or acyclic carbonates, phosphoranes, phosphites, phosphates, phosphonates and heterocycles mentioned above, are those described in WO2007/042471. That document discloses suitable compounds for the present invention selected from
the group of aromatic compounds consisting of 1-acetoxy-2-fluorobenzene, 1-acetoxy-3-fluorobenzene, 1-acetoxy-4-fluorobenzene, 2-acetoxy-5-fluorobenzyl acetate, 4-acetyl-2,2-difluoro-1,3-benzodioxole, 6-acetyl-2,2,3,3-tetrafluorobenzo-1,4-dioxin,
1-acetyl-3-trifluoromethyl-5-phenylpyrazole,
1-acetyl-5-trifluoromethyl-3-phenylpyrazole, benzotrifluoride, benzoyltrifluoroacetone,
1-benzoyl-3-trifluoromethyl-5-methylpyrazole,
1-benzoyl-5-trifluoromethyl-3-methylpyrazole,
1-benzoyloxy-4-(2,2,2-trifluoroethoxy)benzene, 1-benzoyl-4-trifluoromethylbenzene,
1,4-bis(t-butoxy)tetrafluorobenzene, 2,2-bis(4-methylphenyl)hexafluoropropane, bis(pentafluorophenyl) carbonate, 1,4-bis(1,1,2,2-tetrafluoroethoxy)benzene, 2,4-bis(trifluoromethyl)benzaldehyde, 2,6-bis(trifluoromethyl)benzonitrile, difluoroacetophenone, 2,2-difluorobenzodioxole, 2,2-difluoro-1,3-benzodioxole-4-carbaldehyde, 1-[4-(difluoromethoxy)phenyl]ethanone, 3-(3,5-difluorophenyl)-1-propene, fluorobenzophenone, difluorobenzophenone,
1-(2′-fluoro[1,1′-biphenyl]-4-yl)propan-l-one, 6-fluoro-3,4-dihydro-2H-1-benzothiin-4-one, 4-fluorodiphenyl ether, 5-fluoro-l-indanone, 1-(3-fluoro-4-methoxyphenyl)ethanone, fluorophenylacetonitrile,
the group of compounds having an Si—C bond consisting of bis(pentafluorophenyl)dimethylsilane, 1,2-bis[difluoro(methyl)silyl]ethane,
N,O-bis(trimethylsilyl)trifluoroacetamide,
N-(t-butyldimethylsilyl)-N-methyltrifluoroacetamide, t-butyldimethylsilyl trifluoromethanesulphonate, 2-dimethylamino-1,3-dimethylimidazolium trimethyldifluorosiliconate, diphenyldifluorosilane,
the group of compounds having a C═O bond consisting of
bis(1,1,1,3,3,3-hexafluoroprop-2-yl) 2-methylenesuccinate,
bis(1,1,1,3,3,3-hexafluoroprop-2-yl) maleate, bis(2,2,2-trifluoroethyl) maleate,
bis(perfluorooctyl) fumarate, bis(perfluoroisopropyl) ketone,
2,6-bis(2,2,2-trifluoroacetyl)cyclohexanone, butyl 2,2-difluoroacetate, cyclopropyl 4-fluorophenyl ketone, diethyl perfluoroadipate, N,N-diethyl-2,3,3,3-tetrafluoro-propionamide,
the group of compounds having a C=C bond consisting of allyl 1H,1H-heptafluorobutyl ether, trans-1,2-bis(perfluorohexyl)ethylene, (E)-5,6-difluoroocta-3,7-dien-2-one, the group of amines consisting of N,N-diethyl-1,1,2,3,3,3-hexafluoropropylamine as an additive for electrolytes and electrolyte solvents in lithium ion batteries.

The term “difluoroacetophenone” encompasses the isomers with the fluorine substitution in the 2,3-, 2,4-, 2,5-, 2,6-, 3,4- and 3,5-position on the aromatic ring.

The term “fluorobenzophenone” encompasses in particular the isomers 2-fluorobenzophenone and 4-fluorobenzophenone.

The term “difluorobenzophenone” encompasses the isomers with the fluorine substitution in the 2,3′-, 2,3-, 2,4′-, 2,4-, 2,5-, 2,6-, 3,3′-, 3,4′-, 3,4-, 3,5- and 4,4′-position.

The term “fluorophenylacetonitrile” encompasses the isomers with the fluorine substitution in the 2-, 3- and 4-position.

The compounds can be synthesized in a known manner and are also commercially available, for example from ABCR GmbH & Co.KG, Karlsruhe, Germany.

The fluorinated organic compounds mentioned above can be used as the only solvent, i.e. in the form of a single solvent, or they are applied in admixture with one or more organic solvents which are not fluorosubstituted. They can be applied together with linear or cyclic ethers, esters, ketones, saturated or unsaturated alkanes, aromatic hydrocarbons and especially organic carbonates. Alkyl carbonates and alkylene carbonates are the preferred solvent. Often, ethylene carbonate (EC) is comprised in the solvent. The solvent may further contain, low viscosity agents, e.g., ethers like 1,2-dimethoxyethane, tetrahydrofuran, 2-methyltetrahydrofuran, 1,3-dioxolane, 4-methyldioxolane, dimethylcarbonate, ethylmethyl carbonate, diethyl carbonate and any mixtures thereof. Nitriles, e.g., acetonitrile, and t-amyl benzene, and thiosubstituted compounds, for example, ethylene-1,3-dioxolane- 2-thione (ethylene thiocarbonate) are also highly suitable non-fluorinated solvents or additives. The solvent may also additionally contain benzene, fluorobenzene, toluene, trifluorotoluene, xylene or cyclohexane. Lithium bis(oxalato)borate can also be applied. It is no solvent, but an electrolyte salt additive.

Preferred mixtures comprise at least one compound selected from the group consisting of monofluoroethylene carbonate, cis-difluoroethylene carbonate, trans-difluoroethylene carbonate, 4,4- difluoroethylene carbonate, 4-fluoro-4-methyl-1,3-dioxolane-2-one, 4-fluoro-4-ethyl-1,3-dioxolane-2-one, 4-trifluoromethyl-1,3-dioxolane-2-one, 2,2,2-trifluoroethyl-methyl carbonate, 2,2,2-trifluoroethyl-fluoromethyl carbonate, and at least one non-fluorinated organic compound selected from the group consisting of ethylene carbonate, propylene carbonate, dimethyl carbonate, diethyl carbonate, and methyl ethyl carbonate.

The battery solvent contains 0.1 to 100% by weight of the fluorosubstituted organic compound. Often, the fluorinated organic compound is contained in the electrolyte solvent in an amount of equal to or more than 3% by weight. Often, it is contained in an amount of equal to or less than 50% by weight, preferably, equal to or less than 30% by weight.

In batteries which comprise membranes to separate the metallic lithium from the solvent in the cathode compartment, ionic liquids can be applied in a mixture with any of the fluorosubstituted compounds mentioned above. Very suitable ionic liquids are those based on imidazolium, and pyridinium derivatives, but phosphonium or tetraalkylammonium compounds can also be applied. Representative ionic liquids are tosylate, triflate, hexafluorophosphate, bis-(fluorosulfonyl)amide, bis-(trifluoromethylsulfonyl)amide and tetrafluoroborate of 1-ethyl-3-methylimidazolium, and the octyl sulfate of 1-butyl-3-methylimidazolium.

Highly suitable membranes for the Li—S battery are described in U.S. Pat. No. 7,390,591 which is incorporated herein by reference in its entirety for all purposes. These membranes, are highly conductive for ions of the active metal, but are otherwise substantially impervious. They are chemically stable and protect the active metal anode from deleterious reactions with other battery components and decouple the chemical environments of the anode and the cathode. They may be monolithic or be composed of two or more layers.

A first layer, for example, which is in contact with the active metal, may be partially or completely composed of Li₃N, Li₃P, LiI, LiBr, LiC1, LiF and LiPON.

A second layer may be composed of material that is substantially impervious, ionically conductive and chemically compatible with the first material (or its precursor). Suitable materials include glassy or amorphous metal ion conductors, for example, phosphorus-based or oxide based glasses, phosphorus-oxynitride-based glass, selenide-based glass, gallium-based glass, germanium-based glass and boracite glass. Ceramic active metal ion conductors, such as lithium beta-alumina, sodium beta-alumina, Li superionic conductor (LISICON), Na superionic conductor (NASICON) and the like, and glass-ceramic active metal ion conductors are also suitable. Specific examples, e.g., LiPON, are found in U.S. Pat. No. 7,390,591 in column 4, lines 1 to 39.

The layers may further comprise additional components, e.g., polymers, for example, polymer-iodine complexes like polyethylene-iodine, or polymer electrolytes to form flexible composite sheets of material which may be used as second layer of the protective composite. For example, a composite of a Li-ion conducting glass-ceramic material and a solid polymer electrolyte based on polyethyleneoxide-Li salt complexes. Such a material is available from the company Ohara Corp.

The cathode is preferably one of those described in column 15 of U.S. Pat. No. 7,390,591. Suitable cathodes include LiₓCoO₂, LiₓNiO₂, LiₓMn₂O₄, LiFePO₄, AgₓV₂O₅, CuₓV₂O₅, V₂O₅, V₆O₁₃, FeS₂ and TiS₂.

The manufacture of the battery cells is known in the art as indicated in U.S. Pat. No. 7,390,591 in column 15, line 33 to column 16, line 2.

The advantage of the battery cells of the present invention is an improved flame protection and energy density at lower weight and reduced costs.

Another aspect of the present invention concerns an electrolyte solution, comprising
at least one fluorosubstituted organic compound containing at least one heteroatom selected from the group consisting of oxygen, nitrogen, phosphorous, sulfur and silicon,
and wherein the fluorosubstituted organic compound is selected from the group consisting of fluorosubstituted carboxylic acid esters, fluorosubstituted carboxylic acid amides, fluorosubstituted fluorinated ethers, fluorosubstituted carbamates, fluorosubstituted cyclic carbonates, fluorosubstituted acyclic carbonates, fluorosubstituted ethers, perfluoroalkyl phosphoranes, fluorosubstituted phosphites, fluorosubstituted phosphates, fluorosubstituted phosphonates and fluorosubstituted heterocycles,
and at least one compound selected from the group consisting of elemental sulfur and M₂X_{y} wherein M is Na or Li, X is sulfur, and y is 1, 2, 3, 4, 6 or 8. Preferably, M is Li, and M₂X_{y} is Li₂S_{y} and y is 1, 2, 3, 4, 6 or 8. Especially preferably the solution comprises an electrolyte salt selected from the group consisting of LiBF₄, LiCIO₄, LiAsF₆, LiPO₂F₂, LiPF₆ and LiN(CF₃SO₂)₂. The concentration of the electrolyte salt is preferably 1±0.1 molar.

Preferred fluorinated organic compounds are those described above in detail. Fluoroethylene carbonate, cis- and trans-4,5-difluoroethylene carbonate, 4,4-difluoroethylene carbonate, trifluoroethylene carbonate and tetrafluoroethylene carbonate, 4-fluoro-4-methyl-1,3-dioxolane-2-one, 4-fluoro-5-methyl-1,3-dioxolane-2-one, 4-ethyl-4-fluoro-1,3-dioxolane-2one, 5-ethyl-4-fluor-4-ethyl-1,3-dioxolan-2-one and 4,5-dimethyl-4-fluoro-1,3-dioxolane-2-one are especially preferred.

Still another aspect of the present invention concerns the use of a fluorosubstituted organic compound which comprises at least one heteroatom selected from the group consisting of oxygen, nitrogen, phosphorous, sulfur and silicon as sole solvent or in admixture with at least one non-fluorosubstituted solvent, or of lithium bis(oxalato)borate or lithium difluoro(oxalato)borate in the cathode compartment of a lithium oxygen battery, a lithium sulfur battery, or a magnesium-oxygen battery, preferably in a lithium sulfur battery.

Preferably, and especially preferably in a lithium sulfur battery, the fluorosubstituted organic compound is selected from the group consisting of fluorosubstituted carboxylic acid esters, fluorosubstituted carboxylic acid amides, fluorosubstituted fluorinated ethers, fluorosubstituted carbamates, fluorosubstituted cyclic carbonates, fluorosubstituted acyclic carbonates, fluorosubstituted ethers, perfluoroalkyl phosphoranes, fluorosubstituted phosphites, fluorosubstituted phosphates, fluorosubstituted phosphonates and fluorosubstituted heterocycles.

Should the disclosure of any patents, patent applications, and publications which are incorporated herein by reference conflict with the description of the present application to the extent that it may render a term unclear, the present description shall take precedence.

### EXAMPLES

The following examples are intended to explain the invention without limiting it.

FlEC is fluoroethylene carbonate.

### Example 1

### Solutions of Sulfur in FlEC:

50 mg of sulfur were placed into a one-necked PFA-flask. Monofluoroethylene carbonate was added in 10 mL portions. After each addition the mixture was stirred for 10 minutes at 25° C. The sulfur was dissolved after addition of 180 mL.

### Example 2

### Solution of Lithium Sulfide in FlEC:

50 mg of lithium sulfide were placed into a one-necked PFA-flask. Monofluoroethylene carbonate was added in 10 mL portions. After each addition the mixture was stirred for 10 minutes at 25° C. The lithium sulfide was dissolved after addition of 250 mL.

### Example 3

### Preparation of Lithium Polysulfide

100 mg of lithium sulfide were dissolved in 250 mL dry THF. 490 mg Sulfur were added and the mixture was stirred at room temperature for 24 h. The sulfur dissolved, forming lithium polysulfide. After evaporation of the solvent the brown product was dried in vacuo.

### Example 4

### Solution of Lithium Polysulfide in FlEC:

50 mg lithium polysulfide were placed into a one-necked PFA-flask. Monofluoroethylene carbonate was added in 10 mL portions. After each addition the mixture was stirred for 10 minutes at 25° C. The lithium polysulfide was dissolved after addition of 100 mL.

### Example 5

### A Lithium Sulfur Battery

A lithium-sulfur battery is provided which corresponds to the battery type of FIG. 1 of US patent application publication 2009/0061288. It comprises two current collectors. The anode contains lithium. The cathode contains elemental sulfur and Li₂Sₓ (lithium monosulfide and/or lithium polysulfide) and a solvent. The solvent is selected such that it at least partially dissolves the elemental sulfur and the Li₂Sₓ. The battery further contains a substantially non-porous lithium-ion conductive membrane between the anode compartment and the cathode compartment. The membrane is for example a LISICON membrane as available from Ceramatec Inc., Salt Lake City, USA, e.g., a membrane based on Li₁₊ₓAlₓTi₂₋ₓ(PO₄)₃ wherein x is between 0.0 and 0.5. If desired, the membrane can be infused with a lithium salt, e.g., LiPF₆, to conduct lithium ions between anode and the membrane.

The solvent in the cathode compartment is selected from one of the mixtures compiled in Table 1.

TABLE 1
Solvent compositions for the cathode compartment

| | |
|---|---|
| | Solvent composition |
| Example | [% by weight] |

| | | |
|---|---|---|
| 1.1 | EC [85] | F1EC [15] |
| 1.2 | EC [50] | F2EC [50] |
| 1.3 | PC [90] | F2EC [10] |
| 1.4 | PC [50] | F2EC [50] |
| 1.5 | EC [50] | F3EC [25], F4EC [25] |
| 1.6 | PC [70] | F3EC [30] |
| 1.7 | EC [70] | F1DMC [30] |
| 1.8 | EC [80] | 4FPC [20] |
| 1.9 | EC [80] | FMTFEC [20] |
| 2.0 | TG [80] | F1EC [20] |
| 2.1 | TG [80] | F1DMC [20] |

Abbreviations:
EC = ethylene carbonate
PC = propylene carbonate
TG = tetraglyme
F1EC = monofluoroethylene carbonate
F2EC = difluoroethylene carbonate (mixture containing cis-4,5, trans-4,5 and 4,4-isomers)
F3EC = trifluoroethylene carbonate
F4EC = tetrafluoroethylene carbonate
F1DMC = fluoromethyl methyl carbonate
4FPC = 4-fluoro-4-methyl-1,3-dioxolane-2-one
FMTFEC = fluoromethyl 2,2,2-trifluoroethyl carbonate

Operation of the Battery:

If the battery described above is discharged, lithium metal is oxidized at the anode to produce lithium ions and electrons. The electrons pass through a power consuming equipment, and the lithium ions are conducted through the membrane to the cathode where they react with sulfur gradually forming a high polysulfide (e.g., Li₂S₆ or Li₂S₈). The voltage may drop from 2.5 V to 2.1 V.

When the battery is connected to a power source, it will be recharged. Here, the lithium ions from the cathode compartment migrate through the membrane to the anode compartment and are combined with electrons to form elemental lithium. In the cathode compartment, elemental sulfur forms from the Sₓ anions.

### Example 6

### A Lithium Polysulfide Battery

For the battery as described in Example 5, at least one of the solutions of sulfur, lithium sulfide or lithium polysulfide as described in Examples 1, 2 and 4 are mixed in the appropriate amount with the respective other solvent indicated in Table 1 to provide the solution of sulfur, lithium sulfide or lithium polysulfide in the respective solvent as applied in the battery.

1. A Li—S battery comprising an electrolyte solvent which comprises at least one fluorosubstituted organic compound, wherein said at least one fluorosubstituted organic compound comprises at least one heteroatom selected from the group consisting of oxygen, nitrogen, phosphorous, sulfur, and silicon, and wherein the at least one fluorosubstituted organic compound is selected from the group consisting of fluorosubstituted carboxylic acid esters, fluorosubstituted carboxylic acid amides, fluorosubstituted fluorinated ethers, fluorosubstituted carbamates, fluorosubstituted cyclic carbonates, fluorosubstituted acyclic carbonates, fluorosubstituted ethers, perfluoroalkyl phosphoranes, fluorosubstituted phosphites, fluorosubstituted phosphates, fluorosubstituted phosphonates, and fluorosubstituted heterocycles.

2. The battery of claim 1, comprising an anode containing lithium, a cathode containing elemental sulfur,. and current collectors, with said electrolyte solvent which comprises said at least one fluorosubstituted organic compound.

3. The battery of claim 2, comprising an anode compartment and a cathode compartment, and wherein the electrolyte solvent is contained in the cathode compartment.

4. The battery of claim 1 wherein the fluorosubstituted organic compound is selected from the group consisting of monofluoroethylene carbonate, cis-difluoroethylene carbonate, trans-difluoroethylene carbonate, 4,4- difluoroethylene carbonate, trifluoroethylene carbonate, tetrafluoroethylene carbonate, 4-fluoro-4-methyl-1,3-dioxolane-2-one, 4-fluoro-4-ethyl-1,3-dioxolane-2-one, 2,2,2-trifluoroethyl-methyl carbonate, and 2,2,2-trifluoroethyl-fluoromethyl carbonate.

5. The battery of claim 1 wherein the electrolyte solvent further comprises at least one non-fluorinated solvent.

6. The battery of claim 5 wherein the non-fluorinated solvent is selected from the group consisting of alkyl carbonates, alkylene carbonates, and ethers.

7. The battery of claim 6 wherein the non-fluorinated solvent is at least one solvent selected from the group consisting of 1,2-dimethoxyethane, tetraglyme, tetrahydrofuran, 2-methyltetrahydrofuran, 1,3-dioxolane, 4-methyldioxolane, dimethylcarbonate, ethylmethyl carbonate, diethyl carbonate, and any mixtures thereof.

8. (canceled)

9. The battery of claim 1, which further comprises a membrane which is selectively conductive for lithium ions.

10. A cathode compartment of a lithium oxygen battery, a lithium sulfur battery, or a magnesium-oxygen battery, comprising a fluorosubstituted organic compound which comprises at least one heteroatom selected from the group consisting of oxygen, nitrogen, phosphorous, sulfur, and silicon, as sole solvent or in admixture with at least one non-fluorosubstituted solvent, or comprising an organic compound selected from the group consisting of lithium bis(oxalato)borate and lithium difluoro(oxalato)borate.

11. The cathode of claim 10 wherein the fluorosubstituted organic compound is selected from the group consisting of fluorosubstituted carboxylic acid esters, fluorosubstituted carboxylic acid amides, fluorosubstituted fluorinated ethers, fluorosubstituted carbamates, fluorosubstituted cyclic carbonates, fluorosubstituted acyclic carbonates, fluorosubstituted ethers, perfluoroalkyl phosphoranes, fluorosubstituted phosphites, fluorosubstituted phosphates, fluorosubstituted phosphonates, and fluorosubstituted heterocycles.

12. An electrolyte solution, comprising
at least one fluorosubstituted organic compound containin comprising at least one heteroatom selected from the group consisting of oxygen, nitrogen, phosphorous, sulfur, and silicon, wherein the fluorosubstituted organic compound is selected from the group consisting of fluorosubstituted carboxylic acid esters, fluorosubstituted carboxylic acid amides, fluorosubstituted fluorinated ethers, fluorosubstituted carbamates, fluorosubstituted cyclic carbonates, fluorosubstituted acyclic carbonates, fluorosubstituted ethers, perfluoroalkyl phosphoranes, fluorosubstituted phosphites, fluorosubstituted phosphates, fluorosubstituted phosphonates, and fluorosubstituted heterocycles; and
at least one compound selected from the group consisting of elemental sulfur and M₂X_{y}, wherein M is Na or Li, X is sulfur, and y is 1, 2, 3, 4, 6, or 8.

13. The electrolyte solution of claim 12 wherein M is Li.

14. The electrolyte solution of claim 12 wherein M is Li, and wherein the solution further comprises an electrolyte salt selected from the group consisting of LiBF₄, LiCIO₄,LiAsF₆, LiPO₂F₂, LiPF₆, and LiN(CF₃SO₂)₂.

15. The cathode compartment of claim 10, comprising the fluorosubstituted organic compound in admixture with at least one non-fluorosubstituted solvent.

16. The cathode compartment of claim 10, comprising the fluorosubstituted organic compound as sole solvent.

17. The battery of claim 9 wherein the membrane is a monolithic membrane.

18. The battery of claim 9 wherein the membrane consists of two or more layers.

19. The battery of claim 18 wherein a first layer comprises a lithium compound selected from the group consisting of Li₃N, Li₃P, LiI, LiBr, LiCl, LiF, and LiPON.

20. The battery of claim 18 wherein a second layer consists of a material that is substantially impervious and ionically conductive.
